(19) United States
(12) Patent Application Publication
(10) Pub. No.: US 2022/0260471 A1
(43) Pub. Date: Aug. 18, 2022

(54) Three-Dimensional Imaging of a Sample in a Liquid Provision Chamber

(71) Applicant: University of Greenwich, Greenwich, London (GB)

(72) Inventors: Milan ANTONIJEVIC, Greenwich, London (GB); Mike READING, Greenwich, London (GB); Alessandra D'ANGELO, Greenwich, London (GB)

(73) Assignee: University of Greenwich, Greenwich, London (GB)

(21) Appl. No.: 17/623,172

(22) PCT Filed: Jun. 29, 2020
(86) PCT No.: PCT/GB2020/051568

§ 371 Date: Dec. 27, 2021

(30) Foreign Application Priority Data
Jun. 28, 2019 (GB) 1909311.1

(51) Int. Cl. G01N 13/00; G01N 1/38; G01N 33/15; G06T 17/00

(52) U.S. Cl. CPC G01N 13/00; G01N 1/38; G06T 2200/08; G06T 17/00; G01N 2013/006; G01N 33/15

(57) ABSTRACT

An apparatus, method and computer program is described, comprising: rotating a rotatable frame within a liquid provision chamber; providing liquid to a sample on the rotatable frame; obtaining a plurality of images of the sample from a plurality of angles over a first time period; and obtaining a three-dimensional model of the sample based on said plurality of images, wherein the three-dimensional model of the sample comprises quantitative information regarding at least one effect on the sample over the first time period in response to the provision of the liquid.

### FIELD

The present specification relates to an imaging apparatus for obtaining images of a sample.

### BACKGROUND

Samples of pharmaceuticals or other articles may be imaged using imaging devices. There remains a need for further improvements in this field for observing and analysing samples.

### SUMMARY

In a first aspect, this specification describes an apparatus comprising a liquid provision chamber, a plurality of imaging devices, and an output module. The liquid provision chamber may comprises a rotatable frame for holding a sample (e.g. a pharmaceutical product), and a liquid provision mechanism for providing (e.g. conveying or transferring) a liquid (e.g. a solvent, such as water) to the sample. The plurality of imaging devices (e.g. microscopes) may be used for obtaining a plurality of images of the sample from a plurality of angles over a first time period. The output module may be configured to generate or obtain a three-dimensional model of the sample, wherein the three-dimensional model of the sample comprises quantitative information regarding at least one effect on the sample over the first time period in response to the provision of the liquid.

In some examples, the apparatus further comprises an image processing system configured to process (e.g. online or offline processing) the plurality of images to generate said three-dimensional model.

In some examples, the liquid provision mechanism provides the liquid in a controlled flow.

In some examples, the liquid provision mechanism provides liquid on one or more points on the sample.

In some examples, the at least one effect comprises changes in one or more dimensions of the sample.

In some examples, the at least one effect comprises dissolution of at least part of the sample and/or absorption of at least some of the liquid by at least part of the sample.

In some examples, the rotatable frame is inclinable.

In some examples, the liquid provision chamber is a transparent tube or an opaque tube with at least one opening to allow at least one of the plurality of imaging devices to capture images of at least part of the sample.

In some examples, the rotatable frame comprises a reference structure.

In some examples, the reference structure comprises at least one non-uniform pattern and/or one or more protruding elements of known positions and dimensions.

In some examples, the apparatus further comprises an impeller for stirring the liquid.

The apparatus may comprise: at least one processor; and at least one memory including computer program code, the at least one memory and the computer program code configured, with the at least one processor, to cause the performance of the apparatus.

In a second aspect, this specification describes a method comprising: rotating a rotatable frame within a liquid provision chamber; providing liquid to a sample on the rotatable frame; obtaining a plurality of images of the sample from a plurality of angles (for example, using a plurality of imaging devices, such as microscopes) over a first time period; and obtaining a three-dimensional model of the sample based on said plurality of images, wherein the three-dimensional model of the sample comprises quantitative information regarding at least one effect on the sample over the first time period in response to the provision of the liquid.

In some examples, obtaining the three-dimensional model comprises processing said plurality of images using an image processing system.

In some examples, the method further comprises determining said quantitative information.

In some examples, the plurality of images include focussed images of one or more parts of the sample.

In some examples, the plurality of focussed images are combined to provide one or more three-dimensional images, wherein the three-dimensional images are used to obtain said three-dimensional model.

In some examples, providing the liquid to the sample comprises providing a controlled flow of the liquid and/or providing liquid on one or more points on the sample.

In some examples, the at least one effect comprises one or more of: changes in one or more dimensions of the sample; dissolution of at least part of the sample in response to the provision of the liquid; or absorption of at least some of the liquid by at least part of the sample in response to the provision of the liquid.

In some examples, when the liquid is provided on one or more points on the sample, the at least one effect comprises dissolution of the sample at the one or more points.

In some examples, the method further comprises inclining the rotatable frame for draining at least some of the provided liquid.

In some examples, the quantitative information is determined based, at least in part, on positions and/or dimensions of at least part of the sample with reference to a reference structure on the rotatable frame.

In some examples, the method further comprises controlling, using an impeller, a rate at which at least part of the sample dissolves in the liquid.

In some examples, the method further comprises analysing a composition of an analyte over the first time period, wherein the analyte comprises at least part of the provided liquid and/or at least part of the sample.

In some examples, the method further comprises determining a correlation between the analysed composition and said three-dimensional model, wherein the quantitative information includes the determined correlation.

In a third aspect, this specification describes an apparatus configured to perform any method as described with reference to the second aspect.

In a fourth aspect, this specification describes computer-readable instructions which, when executed by a computing apparatus, cause the computing apparatus to perform any method as described with reference to the second aspect.

In a fifth aspect, this specification describes a computer program comprising instructions for causing an apparatus to perform at least the following: rotate a rotatable frame within a liquid provision chamber; provide liquid to a sample on the rotatable frame; obtain a plurality of images of the sample from a plurality of angles (for example, using a plurality of imaging devices, such as microscopes) over a first time period; and obtain a three-dimensional model of the sample based on said plurality of images, wherein the three-dimensional model of the sample comprises quantitative information regarding at least one effect on the sample over the first time period in response to the provision of the liquid.

In a sixth aspect, this specification describes a computer-readable medium (such as a non-transitory computer-readable medium) comprising program instructions stored thereon for performing at least the following: rotate a rotatable frame within a liquid provision chamber; provide liquid to a sample on the rotatable frame; obtain a plurality of images of the sample from a plurality of angles (for example, using a plurality of imaging devices, such as microscopes) over a first time period; and obtain a three-dimensional model of the sample based on said plurality of images, wherein the three-dimensional model of the sample comprises quantitative information regarding at least one effect on the sample over the first time period in response to the provision of the liquid.

### BRIEF DESCRIPTION OF THE DRAWINGS

Example embodiments will now be described, by way of example only, with reference to the following schematic drawings, in which:

FIG. 1 is a block diagram of a system in accordance with an example embodiment;

FIG. 2 is a block diagram of a system in accordance with an example embodiment;

FIG. 3 is a flowchart showing an algorithm in accordance with an example embodiment;

FIGS. 4 to 9 are block diagrams of systems in accordance with example embodiments;

FIGS. 10 and 11 are representations of reference structures in accordance with example embodiments; and

FIGS. 12 to 15 are flowcharts showing algorithms in accordance with example embodiments.

### DETAILED DESCRIPTION

In the description and drawings, like reference numerals refer to like elements throughout.

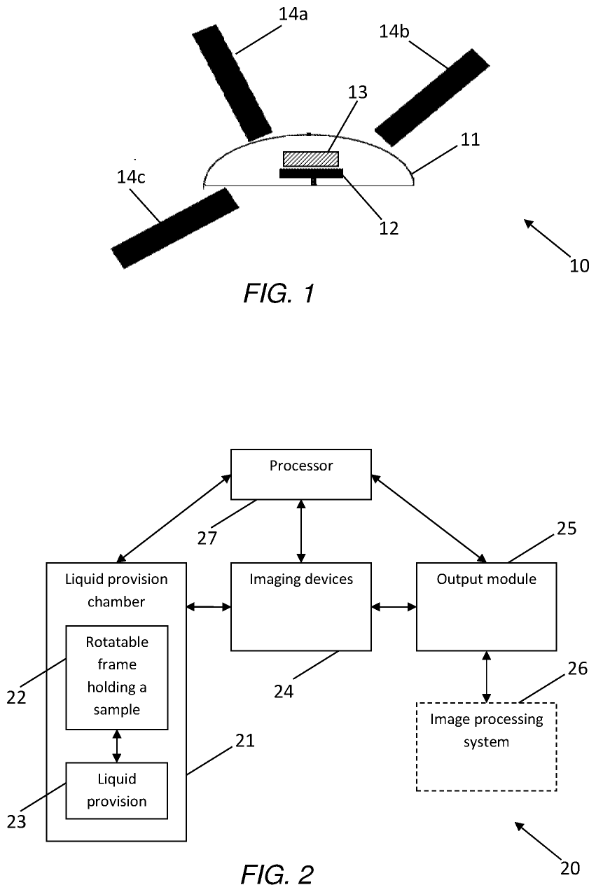

FIG. 1 is a block diagram of an example arrangement of a system, indicated generally by a reference numeral 10, in accordance with an example embodiment. System 10 comprises a liquid provision chamber 11, a rotatable frame 12 holding a sample 13, and a plurality of imaging devices 14 (illustrated as imaging devices 14a, 14b, and 14c). The liquid provision chamber 11 may further comprise a liquid provision mechanism (not shown in this figure) for providing a liquid to the sample 13. The plurality of imaging devices 14 may be configured to obtain a plurality of images of the sample 13 from a plurality of angles. In an example embodiment, the liquid may be a transparent liquid, such that the liquid does not interfere with image capturing of the sample 13 or the quality of the images of the sample 13. For example, the liquid may be water, or may comprise water and/or any other transparent solvent liquid. The liquid may be a solvent such that the sample 13 may be soluble in the liquid.

The system 10 may be arranged for obtaining a plurality of images of the sample, which may then be used for obtaining and analysing various types of information about the sample, for example, quantitative information regarding at least one effect on the sample in response to provision of a liquid. The provision of the plurality of imaging devices 14 may allow the system 10 to obtain images of the sample 13 from a plurality of angles, and may therefore provide detailed information of behaviour of the sample 13 in 3o response to provision of a liquid. Furthermore, placing the sample 13 on the rotatable frame 12 allows the sample 13 to be rotated such that images of a plurality of views of the sample 13 may be captured by one or more of the plurality of imaging devices 14. Details of the operations of system 10 and the analysing of the sample are provided below.

In one example embodiment, the system 10 may be used for analysing dissolution behaviour and/or an absorption behaviour of a sample, for example, a pharmaceutical product. It may be useful to analyse and/or perform simulation of behaviour of a pharmaceutical product, as that may allow better indication of how different medicines and doses of medicines may affect consumers of the pharmaceutical products.

FIG. 2 is a block diagram of a system, indicated generally by a reference numeral 20, in accordance with an example embodiment. System 20 illustrates an example of how the various elements of system 10 may be logically organised. System 20 comprises processor 27, and the processor 27 may be logically arranged with a liquid provision chamber 21, a plurality of imaging devices 24, and an output module 25. The liquid provision chamber 21 comprises a rotatable frame 22 for holding a sample, and a liquid provision mechanism 23 for providing liquid to the sample. The rotatable frame 22 may be rotated, and the liquid provision mechanism 23 may provide liquid to the sample based on signals from the processor 27. Similarly, the imaging devices 24 may capture a plurality of images of the sample from a plurality of angles over a first time period, based on signals received from the processor 27. The output module 25 may be configured to generate or obtain a three-dimensional model of the sample, such that the three-dimensional model of the sample comprises quantitative information regarding at least one effect on the sample over the first time period. The output module 25 may optionally be logically arranged with an image processing system 26. For example, the image processing system 26 may process the plurality of images obtained from the plurality of imaging devices 24. It may be appreciated that the liquid provision chamber 21, the rotatable frame 22, and the plurality of imaging devices 24 may be similar to the liquid provision chamber 11, the rotatable frame 12, and the plurality of imaging devices 14 respectively shown in system 10 of FIG. 1.

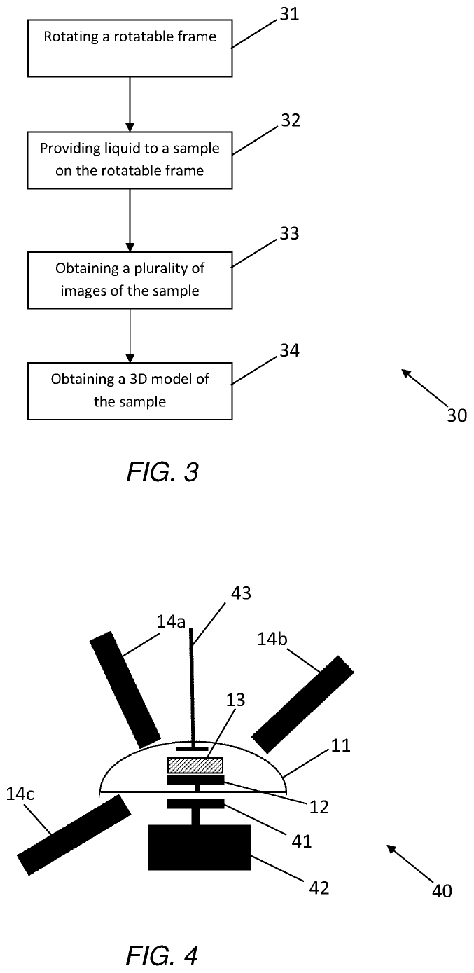

FIG. 3 is a flowchart of an algorithm, indicated generally by the reference numeral 30, in accordance with an example embodiment. At operation 31, a rotatable frame (such as the rotatable frame 12) is rotated in order to rotate a sample. At operation 32, liquid is provided (e.g. conveyed or transferred) to the sample on the rotatable frame by a liquid provision mechanism. At operation 33, a plurality of images of the sample over a first time period is obtained by the plurality of imaging devices. At operation 34, a three dimensional model of the sample is obtained based on the plurality of images obtained at operation 33. The three dimensional model may comprise quantitative information regarding at least one effect on the sample over the first time period in response to the provision of the liquid.

In an example embodiment, the at least one effect on the sample may comprise changes in one or more dimensions of the sample. Alternatively, or in addition, the at least one effect may comprise dissolution of at least part of the sample. Alternatively, or in addition, the at least one effect may comprise absorption of at least some of the liquid by at least part of the sample.

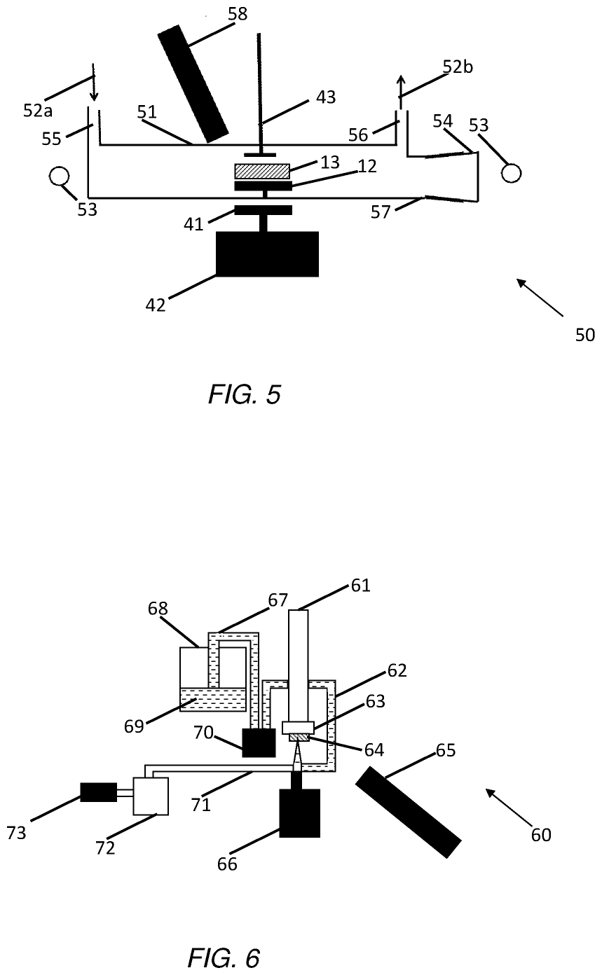

In an example embodiment, at operation 32, the liquid provision mechanism provides the liquid to the sample in a controlled flow. Alternatively, or in addition, the liquid provision mechanism provides liquid at one or more points on the sample. This is described further with reference to FIGS. 5 and 6.

FIG. 4 is a block diagram of an example arrangement of a system, indicated generally by the reference numeral 40, in accordance with an example embodiment. System 40 comprises the liquid provision chamber 11, the rotatable frame 12, the sample 13, and the plurality of imaging devices 14, similar to the elements as described in system 10. System 40 may further comprise a mechanism, such as a magnetic stirrer 41 controlled by a motor 42, which mechanism may be used for rotating the rotatable frame 12. The motor 42 may, for example, be a stepper motor. The mechanism for rotating the rotatable frame 12 may comprise fewer or more elements than the magnetic stirrer 41 and the motor 42, such that the rotatable frame 12 may be rotated in another way. The motor 42 may be controlled using a processor such as the processor 27 for rotating the rotatable frame 12, and therefore also rotating the sample 13 held by the rotatable frame 12. System 40 further comprises an impeller 43 for stirring liquid in a liquid provision chamber 11. For example, stirring the liquid using the impeller 43 may cause the sample 13 to be dissolved in the liquid faster. The impeller 43 may therefore be used for controlling a rate at which at least part of the sample 13 dissolves in the liquid.

FIG. 5 is a block diagram of an example arrangement of a system, indicated generally by the reference numeral 50, in accordance with an example embodiment. System 50 comprises a liquid provision chamber 51, liquid flow 52, at least one light source 53, and a stopper 54. System 50 further comprises one or more elements of system 40 shown in FIG. 4, such as the sample 13, the rotatable frame 12, the magnetic stirrer 41, the motor 42, and the impeller 43. It may be appreciated that one or more of the magnetic stirrer 41, the motor 42, and the impeller 43 may be optional; for example the rotatable frame 12 may be rotated and/or the liquid may be stirred using other means. System 50 shows a liquid provision mechanism for providing a liquid to the sample 13, for example, in the flow 52. Flow 52 may comprise incoming liquid through an opening 55, and outgoing liquid through an opening 56, where the incoming liquid may be illustrated using the arrow 52a and the outgoing liquid may be illustrated using the arrow 52b. The width of openings 55 and 56 may be small relative to the width of the liquid provision chamber 51, as this may allow the liquid to be provided in a controlled flow by the liquid provision mechanism. The system 50 may further comprise a stopper 54, such that the sample 13 may be placed within the liquid provision chamber 51 through opening 57 and then the stopper 54 may be placed for stopping any liquid to flow out of the opening 57, and thus only allowing liquid to flow through the openings 55 and 56. The system 50 may further comprise a plurality of imaging devices 58, which may be similar to the plurality of imaging devices 14. The system 50 may further comprise one or more light sources 53 for providing light when the imaging device 58 captures images of the sample 13. When the liquid is provided in a controlled flow, the dissolution behaviour and/or absorption behaviour of the sample 13 may be analysed in a controlled environment.

It may be appreciated that the system 50 may further comprise other elements required for the functioning of the liquid provision mechanism, which other elements are not shown in FIG. 5 for simplicity.

In an example embodiment, the liquid provision mechanism comprises a peristaltic pump for delivering a liquid into the liquid provision chamber 51. For example, the peristaltic pump may deliver the liquid in a controlled manner, and therefore the rate at which the liquid is provided to the sample 13 may be controlled by controlling the peristaltic pump. For example, the peristaltic pump may comprise a stepper motor, which stepper motor may be rotated at different speeds for controlling the rate at which the peristaltic pump delivers the liquid.

FIG. 6 is a block diagram of an example arrangement of a system, indicated generally by the reference numeral 60, in accordance with an example embodiment. System 60 comprises a magnetic stirrer 61 (similar to the magnetic stirrer 41; motor not shown), a first tube 62, a rotatable frame 63 for holding sample 64, at least one imaging device 65, a linear actuator 66, a second tube 67, a liquid reservoir 68, a liquid 69, a liquid pump 70, a third tube 71, a first vessel 72, and a vacuum pump 73. It may be appreciated that one or more elements (discussed in detail below) of FIG. 6 may be optional, and therefore may be omitted.

System 60 may be representative of a liquid provision mechanism, which liquid provision mechanism provides liquid at one or more points on the sample, such as the sample 64. The sample 64 may be placed on the rotatable frame 63, which rotatable frame may be rotated using the magnetic stirrer 61. For example, the liquid reservoir 68 holds the liquid 69 to be provided to the one or more points on the sample 64. The liquid 69 may flow through the second tube 67 to the liquid pump 70, and the liquid pump 70 may then pump the liquid 69 through the first tube 62 to one or more points on the sample 64. The first tube 62 may, for example, be a coaxial tube system coupled with the linear actuator 66, such that the linear actuator 66 may be used for controlling a positioning of the first tube 62 relative to the sample 64, including the point on the sample 64 that the liquid 69 is provided to, and the distance between an edge of the first tube 62 and a surface of the sample 64. The imaging device 65 may capture images of the sample 64, such that the liquid 69 may be provided to the sample 64 under visual observation using the imaging device 65, and such that the imaging device 65 captures behaviour of the sample 64 in response to the provision of the liquid 69 over a first time period. It may be appreciated that the positioning of the first tube 62 may be controlled using another mechanism, such that the linear actuator 66 may be optional. It may further be appreciated that arranging the sample 64 above an opening of the first tube 62, which opening is used for providing liquid to the sample, may cause direction of a liquid flow to the sample to be upwards (i.e. against gravity), and therefore may prevent unwanted spreading of the liquid through a width and/or depth of the sample 64. This may, in some circumstances, allow better analysing of behaviour of the sample at the one or more points at which the liquid 69 is provided.

In an example embodiment, a composition of an analyte, as drained through the third tube 71, may be analysed over the first time period. The analyte comprises at least part of the provided liquid 69 and/or at least part of the sample 64. The analyte may therefore comprise one or more aliquots of the liquid 69, the sample 64, or a combination (for example a solution) of the liquid 69 and the sample 64. For example, when the liquid 69 is provided to the sample 64, an analyte may be produced when a part of the sample 64 dissolves in a part of the liquid 69, and the analyte may be collected in the first vessel 72 through the third tube 71. It may be possible that no part of the sample 64 dissolves in the liquid 69, in which case the analyte may only comprise the liquid 69. In one example, the composition of the analyte may be analysed under vacuum in the first vessel 72, which vacuum may be created using the vacuum pump 73. The vacuum may allow the composition to be analysed more accurately. The composition of the analyte, as analysed over the first time period, may provide information regarding the dissolution and/or absorption behaviour of the sample 64. For example, if a concentration of particles of the sample 64 in the analyte is increasing at a certain rate, it may be determined that the sample 64 is dissolving in the liquid 69 in the certain rate. Alternatively, or in addition, if the rate of increase in the concentration of particles of the sample 64 is increasing, it may be determined that the sample 64 dissolves at a higher rate in the beginning of the first time period (for example, the beginning of the dissolution process), and at a lower rate towards the end of the first time period (for example, the end of the dissolution process). It should be appreciated that if the sample 64 is only be observed using the images captured, the first vessel 72 and the vacuum pump 73 may be optional.

As such, the dissolution and/or absorption behaviour of the sample 64 may therefore be determined from the image capture over the first time period, from the composition of the analyte, or from both the image capture and the composition of the analyte. There may be various ways in which the sample 64 dissolves in the liquid 69 or absorbs the liquid 69. Further, different points in the sample 64 may also have different dissolution and/or absorption behaviours. The rate of dissolution or absorption may therefore depend on which point of the sample 64 the liquid 69 is provided to, or a layer of the sample 64 that the liquid 69 is provided to. It may be appreciated that different layers of the sample 64 may be exposed to the liquid 69 as outer layers of the sample 64 dissolve and are drained away. The sample 64 may also absorb the liquid 69, and then dissolve in the liquid 69, or a part of the sample 64 may dissolve in the liquid 69, and a part of the sample 64 may absorb the liquid 69. Therefore there may be various possibilities of dissolution or absorption behaviour of the sample, based on the shape, size, and/or composition of the sample 64, and the concentration and/or composition of the liquid 69.

It may be appreciated that one or more elements of the system 60 may be comprised within a liquid provision chamber, such as the liquid provision chamber 11 or 51. A liquid provision mechanism may be different based on the liquid provision chamber. Alternatively, or in addition a liquid provision chamber may be configured to provide a plurality of different liquid provision mechanisms, for example, including providing liquid in a controlled flow (FIG. 5), and providing liquid on one or more points on the sample (FIG. 6).

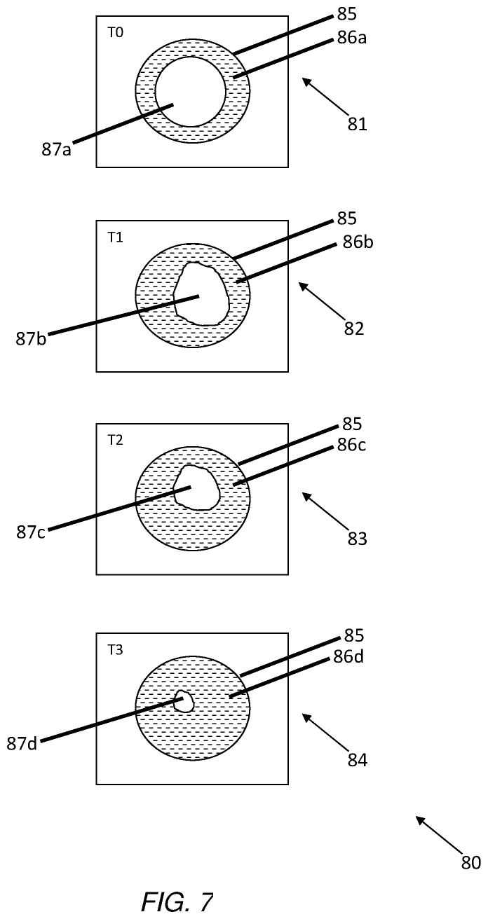

FIG. 7 is a block diagram of a system, indicated generally by the reference numeral 80, in accordance with an example embodiment. System 80 shows dissolution behaviour of a sample, the system 80 comprising example representations 81, 82, 83, and 84 of a sample at different time instances T0, T1, T2, and T3 over a first time period. The representations 81, 82, 83, and 84 may be images as captured by an imaging device over the first time period. The sample, for example sample 87, may be held on a rotatable frame 85. A liquid 86 may be provided to the sample 87. As shown in the representations 81 to 84, the size and/or composition of the sample 87 may change, for example from sample 87a to sample 87b to sample 87c to sample 87d. Similarly, the volume, concentration and/or composition of the liquid 86 may change, for example from liquid 86a to liquid 86b, to liquid 86c, to liquid 86d. For example, at time T0, the sample size may be represented by the sample 87a. Time T0 may be at the beginning of the first time period. The sample size at sample 87a may be an initial sample size before any part of the sample has absorbed any liquid or has dissolved in any liquid. At time T1, the sample size may be represented by the sample 87b. It can be seen that the sample size in sample 87b is different from the sample size in sample 87a. The sample size may be smaller in sample 87b as at least some part of the sample may have dissolved in the liquid 86 from the time T0 to the time T1. Similarly, the sample size continues to become smaller in samples 87c and 87d, as more parts of the sample dissolve in the liquid 86. As the sample dissolves in the liquid, the liquid concentration and composition may also change. Therefore, liquid 86a, 86b, 86c, and 86d may have different compositions. For example, at time T0, the liquid 86a may have little or no pails of the sample; at time T1, the liquid 86b may have some more concentration of the sample compared to the liquid 86a, as some parts of the sample has dissolved in the liquid; at time T2, the liquid 86c may have some more concentration of the sample compared to the liquid 86b, as some more parts of the sample have dissolved in the liquid; and at time T3, the liquid 86d may have the most concentration of the sample compared to the liquids 86a, 86b, and 86c, as most parts of the sample have dissolved in the liquid. It may be appreciated that the system 80 provides example representations of the dissolution behaviour of the sample, such that the sample may dissolve in any other way, and the dissolution behaviour may not be limited to that shown in system 80.

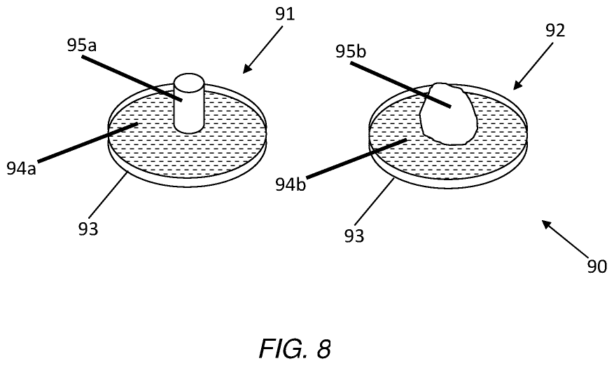

FIG. 8 is a block diagram of a system, indicated generally by the reference numeral 90, in accordance with an example embodiment. System 90 shows absorption behaviour of a sample, the system 90 comprising example representations 91 and 92 of a sample at different time instances T0 and T1 over a first time period. The representations 91 and 92 may be images as captured by an imaging device over the first time period. The sample, for example sample 95, may be held on a rotatable frame 93. A liquid 94 may be provided to the sample 95. As shown in the representations 91 and 92, the shape, size, and/or composition of the sample 95 may change, for example from sample 95a to sample 95b. Similarly, the volume, concentration, and/or composition of the liquid 94 may change, for example from liquid 94a to liquid 94b. For example, the at least some part of the sample 95 may absorb at least some part of the liquid 94. As such, the absorption of the liquid 94 by the sample 95 may cause the shape and size of the sample to change from sample 95a to sample 95b.

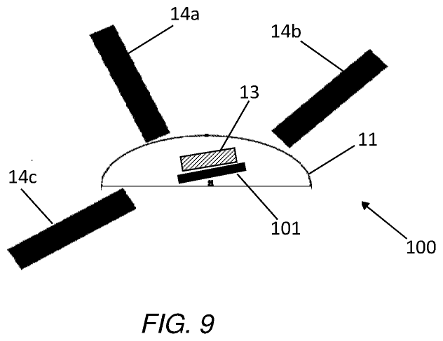

In one example, the absorption of the liquid by the sample may further be determined based on the change in volume of the liquid from time T0 to time T1. FIG. 9 is a block diagram of a system, indicated generally by the reference numeral 100, in accordance with an example embodiment. System 100 comprises some of the elements of system 10 including the liquid provision chamber 11, the sample 13, and the plurality of imaging devices 14. The system 100 further comprises a rotatable frame 101. The rotatable frame 101 may be inclinable. The inclination of the rotatable frame 101 may be controlled in order to drain any remaining liquid that is not used up in dissolution or absorption by the sample 13. For example, with reference to FIG. 7, the rotatable frame 85 may be similar to the rotatable frame 101, such that the rotatable frame 85 may be inclinable. The liquid 86d at time T3 may be drained by inclining the rotatable frame 85, and the collected liquid 86d may then be analysed for determining dissolution behaviour of the sample, for example, by determining the mass, volume, or composition of the liquid 86d. In another example, with reference to FIG. 8, the rotatable frame 93 may be similar to the rotatable frame 101, such that the rotatable frame 93 may be inclinable. The liquid 94b at time T1 may be drained by inclining the rotatable frame 93, and the collected liquid 94b may then be analysed for determining absorption behaviour of the sample, for example, by determining the mass, volume, or composition of the liquid 94b.

In an example embodiment, the liquid provision chamber (for example the liquid provision chambers 11 or 51) may comprise a transparent tube. Alternatively, or in addition, the liquid provision chamber may comprise an opaque tube with at least one opening to allow at least one of the plurality of imaging devices 14 to capture images of at least part of the sample. The plurality of openings may therefore be positioned to allow clear imaging by the plurality of imaging devices 14. The plurality of openings may be covered with transparent material, such that the liquid may not exit the liquid provision chamber through any of the plurality of openings, and the imaging devices are able to capture images of at least part of the sample.

As discussed earlier, the plurality of images of the sample may be used for obtaining and analysing various types of information about the sample, for example, quantitative information regarding at least one effect on the sample in response to provision of a liquid. The rotatable frame may comprise one or more reference structures, such that the images of the sample may provide quantitative information based on the positioning, shape, or size of one or more pails of the sample relative to the reference structures. The dimensions and other quantitative details of the reference structures may be known, such that the known quantitative details of the reference structures may be used for obtaining quantitative information regarding the behaviour of the sample in response to provision of a liquid.

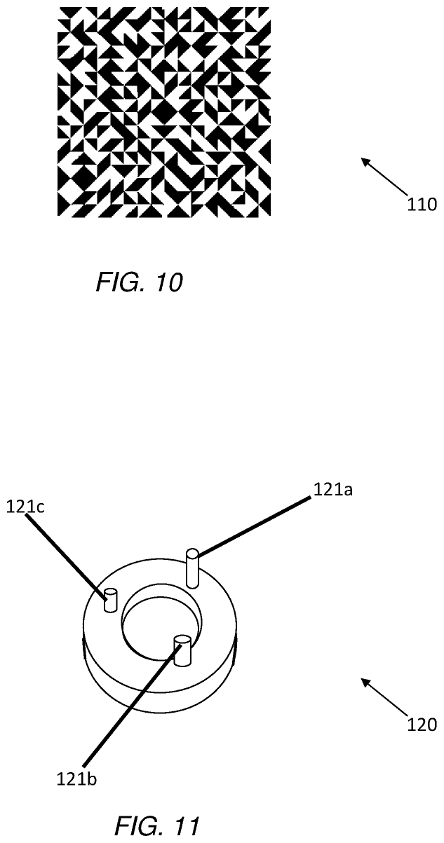

FIG. 10 is a representation of a reference structure, indicated generally by the reference numeral 110, in accordance with an example embodiment. The rotatable frame, such as the rotatable frames 12 and 101, may comprise a reference structure 110, which reference structure 110 comprises at least one non-uniform pattern. The non-uniform pattern may have different colours in different pails (the different colours are not shown in the figure). As such, the pattern, along with the different colours allows the non-uniform pattern to be a non-repeating pattern.

FIG. 11 is a representation of a reference structure, indicated generally by the reference numeral 120, in accordance with an example embodiment. The rotatable frame, such as the rotatable frames 12 and 101, may comprise the reference structure 120, which reference structure 120 comprises one or more protruding elements 121 of known positions and dimensions. In an example embodiment, the rotatable frame may comprise both of the reference structures 110 and 120, such that the surface of the rotatable frame comprises the non-uniform pattern of the reference structure 110, and also comprises the protruding elements 121 of the reference structure 110. The dimensions (such as diameter, height, width, etc.) of the protruding elements 121a, 121b and 121C may be different from each other, and may be known. For example, the protruding element 121a has the highest height, and the protruding element 121C has the lowest height.

With reference to FIG. 3, the plurality of images of the sample may be used for obtaining a three-dimensional model of the sample. In an example embodiment, the three-dimensional model is obtained from the plurality of images by using a photogrammetry technique. In the photogrammetry technique, the plurality of images may be overlapped to create the three-dimensional model, which overlapping may be facilitated with any irregularities in the image. However, since the sample may not have many irregularities (for example, a white tablet may be a uniform object), the reference structures of the rotatable frame may assist with performing successful photogrammetry. The non-uniform pattern and the protruding elements may assist the recognition of overlapping areas, and therefore the plurality of images may be aligned accurately. The non-uniform pattern may assist in horizontal alignment (for example, in the horizontal plane), and the protruding elements may assist in vertical alignment (for example in the vertical plane). Therefore a three-dimensional model may comprise quantitative information of the sample in all three dimensions.

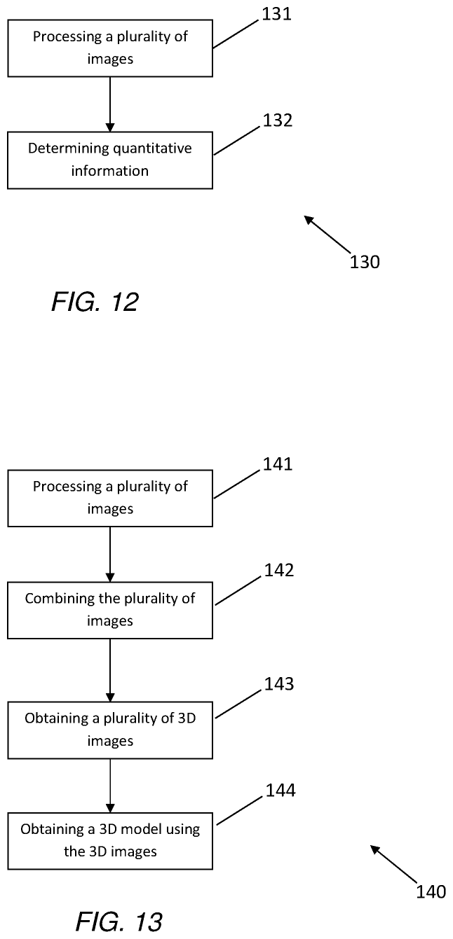

FIG. 12 is a flowchart of an algorithm, indicated generally by the reference numeral 130, in accordance with an example embodiment. The algorithm 130 starts with operation 131 for processing a plurality of images of the sample, as obtained from imaging devices described above. At operation 132, quantitative information of the sample is determined based on the processed images.

For example, with reference to FIG. 2, processing the plurality of images may be performed at the image processing system 26. The processing of the plurality of images may comprise a number of image processing techniques, such as enhancing or illuminating one or more of the plurality of images. The image processing may be performed online (e.g. substantially in real time as the images of the sample are captured), or may be performed offline (e.g. after the plurality of images of the sample are captured). Further steps of the image processing are provided in FIG. 13.

FIG. 13 is a flowchart of an algorithm, indicated generally by the reference numeral 140, in accordance with an example embodiment. At operation 141, the plurality of images may be processed, as described in operation 131. The plurality of images may include focussed images of one or more parts of the sample. At operation 142, at least two of the plurality of images may be combined to obtain a plurality of three-dimensional images at operation 143. Each of the plurality of three-dimensional images may be obtained by combining images from a single time instance, or from more than one consecutive time instances. As such, each three-dimensional image may represent the sample from all three dimensions at a given time. The plurality of three-dimensional images may represent the sample from all three dimensions at a plurality of time instances over the first time period. At operation 144, a three-dimensional model of the sample may be obtained based on the plurality of three-dimensional images. The three-dimensional model may provide a continuous time series of how the sample behaves over the first time period in response to provision of liquid.

In an example embodiment, quantitative information regarding at least one effect on the sample may be obtained (such as, in operation 132) from the three-dimensional model based position, dimensions, size, and/or shape of the sample relative to one or more reference structures of the rotatable frame, which reference structures are captured in the images.

In an example embodiment, as each of the plurality of images obtained from the imaging devices may be focussed images of one or more parts of the sample, the three-dimensional images (obtained by combining the images) may comprise a high-resolution image where all or a majority of parts of the sample may be focussed. If a single image of the sample is captured, one or more parts of the sample may be out of focus. As the three-dimensional images obtained in operation 143 are images where all or at least a majority of the parts of the sample are in focus, an out-of-focus fraction of the image may be minimized. Furthermore, as the sample dissolves, the size of the sample may become smaller, which may cause the sample to become out of focus of one or more imaging devices. As a plurality of imaging devices is used, a plurality of planes of focus may be available at the respective plurality of imaging devices. The plurality of imaging devices may be positioned radially (for example in a circle surrounding the sample). When the sample rotates on the rotatable frame, at least one of the plurality of imaging devices may capture a focussed image of the sample. In an example embodiment, the plurality of images that are combined (for example in operation 142 of FIG. 13) may not contain out-of-focus images. For example, the out of focus images may be removed using Shannon Entropy technique, which technique may be used for measuring lack of focus of the images. As such, the remaining images (excluding the out-of-focus images) may be combined in operation 142 for obtaining the three-dimensional model. In another example, one or more focal planes of respective one or more of the plurality of imaging devices may be moved within a range (for example, using a linear actuator). The focal planes may be moved using a z-stalking technique, such that most or all of the plurality of the images are focussed images.

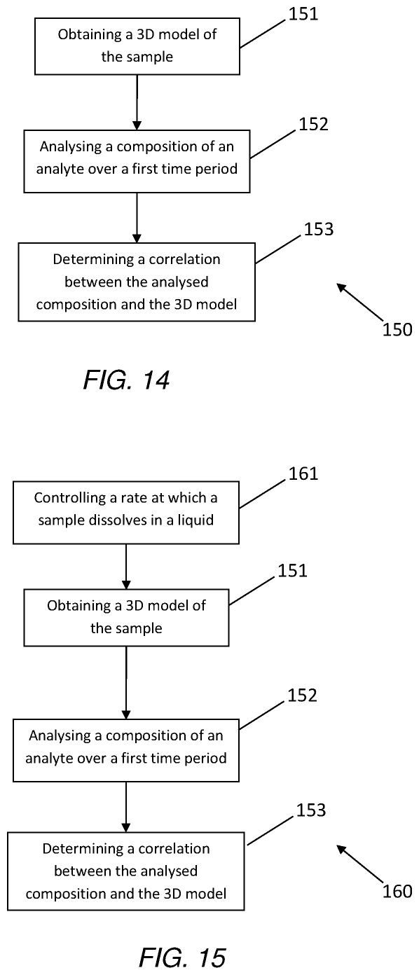

FIG. 14 is a flowchart of an algorithm, indicated generally by the reference numeral 150, in accordance with an example embodiment. At operation 151, a three-dimensional model of the sample over a first time period may be obtained (as shown in algorithm 140). At operation 152, a composition of an analyte may be analysed over the first time period (as discussed in FIG. 6). At operation 153, a correlation between the analysed composition and the three-dimensional model may be determined. For example, the quantitative information determined in algorithm 130 may comprise the determined correlation. In one example, the quantitative information related to the correlation may comprise dissolution dynamics of the sample. For example, the three dimensional model may provide information regarding a rate of dissolution, dissolution of different parts of the sample, and the like. The composition of the analyte at different time instances may also provide information regarding the rate of dissolution by analysing the different concentrations of the sample in the liquid at different time instances. The correlation may be determined in order to provide more accurate information for at least one effect on the sample in response to the provision of a liquid.

FIG. 15 is a flowchart of an algorithm, indicated generally by the reference numeral 160, in accordance with an example embodiment. At operation 161, a rate at which the sample dissolves in a liquid may be controlled. For example, the rate may be controlled using an impeller, such as impeller 43 shown in FIG. 4. The impeller 43 may be stirred for increasing the rate of dissolution. In another example, the rate of the dissolution may be reduced by introducing a non-solvent liquid (for example, glycerine, silicon oil, or any other transparent non-solvent liquid) in the liquid. Operations 151, 152, and 153 may then be performed as described in algorithm 150.

The methods, systems, and apparatus described above may be utilized for various applications. For example, the sample may be a pharmaceutical drug, such as a tablet. Analysing dissolution behaviour of the tablet may provide information about how the sample may behave in a stomach of a patient, and one or more properties (such as size, dosage, concentration of active elements) of the tablet may be adjusted accordingly. The dissolution and/or absorption behaviour of similar drugs from different manufacturers may also be compared, such that it may be determined whether the drugs behave similarly or differently in a patient's stomach. Similarly, counterfeit drugs may also be identified, as they may have different dissolution and/or absorption behaviours compared to an original or legally approved drug. As such, the liquid used for analysing the dissolution and/or absorption behaviour may be similar to liquids present in patients' stomach. The liquid may comprise one or a plurality of solvents. Analysing the absorption behaviour may be useful for determining absorption by a wafer, for example a buccal medicine that may be placed internally in the mouth of a patient, and may require absorption by inner tissues of the mouth.

If desired, the different functions discussed herein may be performed in a different order and/or concurrently with each other. Furthermore, if desired, one or more of the above-described functions may be optional or may be combined. Similarly, it will also be appreciated that the flowcharts of FIGS. 3 and 12 to 15 are examples only and that various operations depicted therein may be omitted, reordered and/or combined.

It will be appreciated that the above described example embodiments are purely illustrative and are not limiting on the scope of the invention. Other variations and modifications will be apparent to persons skilled in the art upon reading the present specification. Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

Although various aspects of the invention are set out in the independent claims, other aspects of the invention comprise other combinations of features from the described example embodiments and/or the dependent claims with the features of the independent claims, and not solely the combinations explicitly set out in the claims.

It is also noted herein that while the above describes various examples, these descriptions should not be viewed in a limiting sense. Rather, there are several variations and modifications which may be made without departing from the scope of the present invention as defined in the appended claims.

1. An apparatus comprising:
a liquid provision chamber; wherein the liquid provision chamber comprises:
a rotatable frame for holding a sample; and
a liquid provision mechanism for providing a liquid to the sample;
a plurality of imaging devices for obtaining a plurality of images of the sample from a plurality of angles over a first time period; and
an output module configured to generate or obtain a three-dimensional model of the sample, wherein the three-dimensional model of the sample comprises quantitative information regarding at least one effect on the sample over the first time period in response to the provision of the liquid.

2. An apparatus as claimed in claim 1, further comprising an image processing system configured to process the plurality of images to generate said three-dimensional model.

3. An apparatus as claimed in claim 1, wherein the liquid provision mechanism provides the liquid in a controlled flow.

4. An apparatus as claimed in claim 1, wherein the liquid provision mechanism provides liquid on one or more points on the sample.

5. An apparatus as claimed in claim 1, wherein the at least one effect comprises changes in one or more dimensions of the sample.

6. An apparatus as claimed in claim 1, wherein the at least one effect comprises dissolution of at least part of the sample and/or absorption of at least some of the liquid by at least part of the sample.

7. An apparatus as claimed in claim 1, wherein the rotatable frame is inclinable.

8. The apparatus of claim 1, wherein the liquid provision chamber is a transparent tube or an opaque tube with at least one opening to allow at least one of the plurality of imaging devices to capture images of at least part of the sample.

9. The apparatus of claim 1, wherein the rotatable frame comprises a reference structure.

10. The apparatus of claim 9, wherein the reference structure comprises at least one non-uniform pattern and/or one or more protruding elements of known positions and dimensions.

11. The apparatus of claim 1, further comprising an impeller for stirring the liquid.

12. A method comprising:
rotating a rotatable frame within a liquid provision chamber;
providing liquid to a sample on the rotatable frame;
obtaining a plurality of images of the sample from a plurality of angles over a first time period; and
obtaining a three-dimensional model of the sample based on said plurality of images, wherein the three-dimensional model of the sample comprises quantitative information regarding at least one effect on the sample over the first time period in response to the provision of the liquid.

13. A method as claimed in claim 12, wherein obtaining the three-dimensional model comprises processing said plurality of images using an image processing system.

14. A method as claimed in claim 12, further comprising determining said quantitative information.

15. A method as claimed in claim 12, wherein the plurality of images include focussed images of one or more parts of the sample.

16. A method as claimed in claim 15, wherein the plurality of focussed images are combined to provide one or more three-dimensional images, wherein the three-dimensional images are used to obtain said three-dimensional model.

17. A method as claimed in claim 12, wherein providing the liquid to the sample comprises providing a controlled flow of the liquid and/or providing liquid on one or more points on the sample.

18. A method as claimed in claim 12, wherein the at least one effect comprises one or more of:
changes in one or more dimensions of the sample;
dissolution of at least part of the sample in response to the provision of the liquid; and
absorption of at least some of the liquid by at least part of the sample in response to the provision of the liquid.

19. A method as claimed in claim 18, wherein when the liquid is provided on one or more points on the sample, the at least one effect comprises dissolution of the sample at the one or more points.

20. A method as claimed in claim 12, further comprising inclining the rotatable frame for draining at least some of the provided liquid.

21. A method as claimed in claim 12, wherein the quantitative information is determined based, at least in part, on positions and/or dimensions of at least part of the sample with reference to a reference structure on the rotatable frame.

22. A method as claimed in claim 12, further comprising controlling, using an impeller, a rate at which at least part of the sample dissolves in the liquid.

23. A method as claimed in claim 12, further comprising analysing a composition of an analyte over the first time period, wherein the analyte comprises at least part of the provided liquid and/or at least part of the sample.

24. A method as claimed in claim 23, further comprising determining a correlation between the analysed composition and said three-dimensional model, wherein the quantitative information includes the determined correlation.

25. A computer program comprising instructions for causing an apparatus to perform at least the following:
rotate a rotatable frame within a liquid provision chamber;
provide liquid to a sample on the rotatable frame;
obtain a plurality of images of the sample from a plurality of angles over a first time period; and
obtain a three-dimensional model of the sample based on said plurality of images, wherein the three-dimensional model of the sample comprises quantitative information regarding at least one effect on the sample over the first time period in response to the provision of the liquid.
